SEQUENCE LISTING (7 sequences)

SEQ ID NO: 1 - DNA, 20 bp, Escherichia coli
ggaaacagctatgaccatga

SEQ ID NO: 2 - DNA, 20 bp, Escherichia coli
gcaggcttctgcttcaatca

SEQ ID NO: 3 - DNA, 20 bp, Escherichia coli
tcatggtcatagctgtttcc

SEQ ID NO: 4 - DNA, 20 bp, Escherichia coli
ggaaacagctatgaccatga

SEQ ID NO: 5 - DNA, 20 bp, Escherichia coli
gcaggcttctgcttcaatca

SEQ ID NO: 6 - DNA, 20 bp, Escherichia coli
tcatggtcatagctgtttcc

SEQ ID NO: 7 - DNA, 20 bp, Escherichia coli
ggaaacagctatgaccatga

(19) United States
(12) Patent Application Publication
(10) Pub. No.: US 2003/0148304 A1
(43) Pub. Date: Aug. 7, 2003

(54) Method for immobilisation

(76) Inventors: Zicai Liang, Sundbyberg (SE); Anil Kumar, Farsta (SE)

Correspondence Address: Stanley R. Moore, Esq., Jenkens & Gilchrist, P.C., Suite 3200, 1445 Ross Avenue, Dallas, TX, 75202-2799, US

(21) Appl. No.: 10/165,642

(22) Filed: Jun. 7, 2002

Related U.S. Application Data
(63) Continuation of application No. PCT/SE00/02446, filed on Dec. 6, 2000.
(60) Provisional application No. 60/170,208, filed on Dec. 10, 1999.

(30) Foreign Application Priority Data
Dec. 9, 1999 (SE) 9904506-4

(51) Int. Cl. C12Q 1/68; B05D 3/00; C07H 21/02; C07H 21/04; C12M 1/34; C07K 14/00

(52) U.S. Cl. 435/6; 435/287.2; 427/2.11; 536/23.1; 530/350

(57) ABSTRACT

A method for immobilisation of nucleic acids onto glass and silicon surfaces is described, wherein said nucleic acids are immobilised onto unmodified glass and other silicon surfaces. A new class of modified nucleic acids, namely silanised nucleic acids, and methods of preparing such modified nucleic acids, as well as a method for producing DNA chips of various density with only end-attachment of DNA applied, are also described.

### TECHNICAL FIELD OF THE INVENTION

The present invention relates generally to inimobilisation of nucleic acids (oligonucleotides, DNA, RNA, anid peptide nucleic acid (PNA)) onto glass and silicon surfaces after manual or automated deposition, and more particularly to immobilisation of silanised nucleic acids.

### TECHNICAL BACKGROUND

Solid phase nucleic acid hybridisation has been used in a wide variety of applications including monitoring gene expression, polymorphism analysis, disease screening and diagnostics, nucleic acid sequencing, anid genome analysis. A number of different substances have been tested as the solid support for nucleic acid immobilisation, but lass and silicon remain to be the most favoured supporting materials for DNA anid oligonucleotide chips (microarrays). In general there are two ways by which nucleic acids molecules can be established on the glass surface: Direct on-surface synthesis; and immobilisation of pre-fabricated nucleic acids. On-chip synthesis of oligonucleotides by photolithographic DNA synthesis is by far the most efficient method of generating high-density oligonucleotide chips on a lass surface. But it also has its practical limitations in terms of flexibility, accessibility anid affordability. Imimobilisation of pre-fabricated nucleic acids, on the other hand, offers excellent flexibility that can accommodate most research and clinical applications. For making chips of medium or low complexity, imrmobilisation can provide a chip production speed that is much higher than for photolithographic synthesis. Such immobilisation technologies are becoming, widely available and, more importantly, much more affordable for most researchers anid clinicians. Central to the immobilisation technologies is the development of efficient chemistries for covalent attachment of nucleic acids onto glass and silicon surfaces. A great number of attachment methods have been disclosed, which vary widely in chemical mechanisms, ease of use, probe density and stability. Silane derivatives constitute a large family of chemicals with a wide range of chemical properties, especially in terms of reactivity towards other chemicals. They have been widely used to modify glass surfaces to accommodate the attachment of differently modified, or even unmodified, biomolecules.

Thus, in all previously reported attachment methods, glass and silicon surfaces have been extensively modified in order to achieve best reactivity against correspondingly modified, or unmodified, nucleic acids and to minimise background signals generated by such surface modifications. Such modifications have been carried out using various silylating reagents carrying reactive groups or being able to react with chemicals carrying reactive goups. The reactive groups thus immobijised on a glass surface will then react with cognate reactive groups that are constructed on the nucleic acids so that the nucleic acids can be covalently immobilised. One shortcoming of such procedures is that the modified surface may obtain affinity towards the probes used to detect the immobilised nucleic acids and give rise to extra undesired signals. A second shortcoming of such procedures is that the actual immobilisation reaction (between the glass-bound reactive group and the nucleic acid-bound cognate reactive group) is always under un-optimised conditions, especially in DNA array applications where a minute amount of nucleic acid solution (10 pl-200 nl) is deposited as tiny droplets on a glass surface, which will be dried up within seconds. Because of sampledrying during arraying, the arrays have to be incubated in a humidified chamber for over 12 hours in order to allow the reaction to take place.

### SUMMARY OF THE PRESENT INVENTION

The present invention provides a new method of immobilisation of nucleic acids onto untreated glass and silicon surfaces, in which a new class of modified nucleic acid. namely silanised nucleic acids, is used. The inventive nucleic acid modification enables the modified molecules to be covalently attached directly onto any untreated glass and silicon surface. The method of the invention is based upon procedures to covalently conjugate an active silyl moiety onto nucleic acids in solutions, to form the silanised nucleic acids, and subsequently to apply such silanised molecules onto glass surface for instant immobilisation by means of the active silyl moiety. All steps of the inventive immobilisation procedure can be monitored quantitatively and closely controlled. It has also been found that this procedure has the capability to generate a very even and intense monolayer of oligonucleotides that are accessible for hybridisation on the unmodified glass surface where background is very low. The method of the invention provides a simple, yet very efficient, solution to the previously mentioned innnobilisation problems associated with manual or automated deposition of pre-fabricated nucleic acids.

Accordingly, a chemistry has been developed to allow simultaneous deposition and covalent linking of nucleic acids onto umnodified glass and other silicon surfaces. In the examples below, three preferred pathways will be demonstrated, which can be used to generate such molecules in either aqueous solutions or an organic solvent. The immobilisation method can be used to produce nucleic acid chips of various density and results in only end-attachment of the nucleic acids applied. While silanes in the prior art have been widely used to modify glass surfaces to accommodate the attachment of differently modified biomolecules, this usage has also strictly limited the number of differently modified nucleic acids that can be immobilised onto one and the same glass chip. The present invention, on the other hand, enables a single untreated glass chip to accommodate an unlimited number of differently modified nucleic acids, after they have been conjugated to their cognate silanes according to the present invention.

Furthermore, the procedure for conjugating a silyl moiety to nucleic acid molecules according to the invention is simple and rapid. With both manual and automated arraying enabled and good availability of reagents (normal glass slides, silylating reagents and commercially available modified oligonucleotides), silanized nucleic acids would allow anyone to practice array experiments easily on glass substrates. By using the vast knowledge of silane-related reactions in the method of preparing silanised nucleic acids according to the invention, these will provide a universal platform for arrayed deposition of biomolecules on glass or silicon surfaces.

### BRIEF DESCRIPTION OF THE ATTACHED DRAWINGS

FIG. 1 depicts three different pathways (A, B, and C) for silanisation of nucleic acids of the present invention.

FIG. 2A illustrates silanised Lac-Thio oligonucleotide arrayed manually, and 2B with an automated arrayer, respectively. The hybridisation signal from a manually spotted chip during repeated stripping and hybridisation is quantified in 2C.

In FIG. 3 the specificity of silanisation reaction towards thiol labelled oligonucleotides and unlabelled oligonucleotides is compared.

FIG. 4A illustrates direct immobilisation of thiol labelled cDNA. The hybridisation results are shown for manual spotting in 4B and C, and with an automated arrayer in 4D, respectively.

FIG. 5 depicts oligonucleotide monolayers formed under coverslips using silanised oligonucleotides in dimethyl sulphoxide and its quantification.

FIG. 6A shows hybridisation results of chips made with silanised acrylic oligonucleotiedes by automated arraying, and 6B and C, by manually spotting

FIG. 7 demonstrates that chips made with silanised nucleic acids can subsequently be treated to acquire desired surface properties while having little effect on the performance of the immobilised nucleic acids.

### DETAILED DESCRIPTION OF THE PRESENT INVENTION

According to the present invention silanised nucleic acids can be prepared by several chemical pathways. Silanised nucleic acids have heretofore never been investigated in the art of nucleic acid modifications, presumably because of the common general knowledge that the silyl moiety is not stable in aqueous solutions at a high pH. However, it has now been found that by employing an organic solvent or aqueous solution at low pH, the synthesis of silanised nucleic acids is feasible and that these molecules are stable for weeks under proper storage conditions (−20° C. when not in use).

Thus, the present invention is based upon the finding that a silane-nucleic acid conjugate can be constructed and react with a glass surface at similar efficiencies as a normal silane, and that different silylating reagents and correspondingly modified nucleic acids can be conjugated with each other in their respectively optimised solutions, and then be deposited pointedly onto glass chips. Such an approach allows both the conjugation and glass immobilisation reactions to be accomplished in the shortest time. This procedure has also been found to incur minimal background signals, as compared to the prior art immobilisation methods, in which the surface modifying reagents has been found to give rise to background signals in surface areas of the substrate surface with no target molecules immobilised.

The nucleic acid used in the present invention can for example be modified with a group selected from thio, dithio, amino, aldehydo, keto, hydrazo, acrylic, hydrazino, halo, or carboxyl.

According to the present invention different silanes (silylating reagents) can be conjugated to nucleic acids. In order to achieve the conjugation, the silylating reagents should carry a group which can be reacted with the label of the nucleic acids, as will be shown in further detail in the Examples. Sometimes, a coupling reagent will be needed in order to initiate the silylation reaction. Such a coupling reagent will not become part of the final conjugated molecule. Alternatively, a bifunctional linker or spacer molecule could be used, which is reactive to both the label of the nucleic acid, and the reactive groups of the silylating reagents, thereby linking the two molecules together, thus establishing the desired conjugation.

Three different suitable pathways have been demonstrated experimentally for the synhesis of silanised nucleic acids:

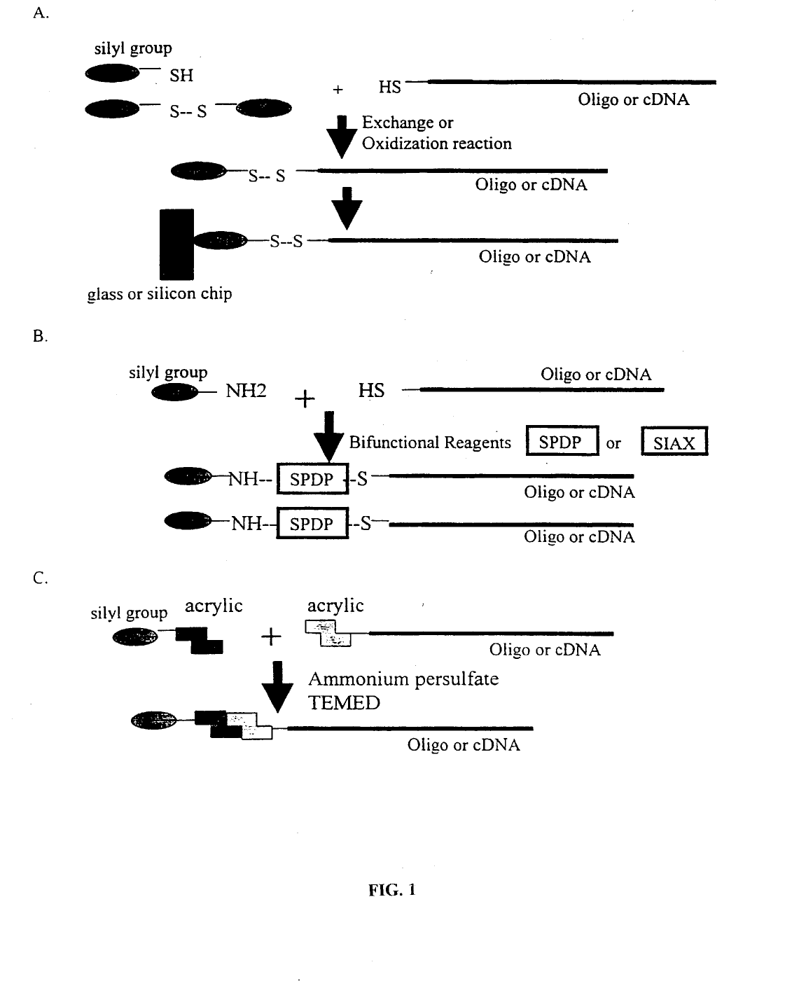

Firstly, with reference to FIG. 1A, (3-mercaptopropyl)-trimehoxysilane, which is believed to provide the least non-specific binding of oligonucleotides, is conjugated to a thiol labelled oligonucleotide. The thiol group of the silane can slowly undergo spontaneous oxidisation to form intermolecular disulphide bonds. The conjugation of (3-mercaptopropyl)-trimethoxysilane with thiol labelled oligonucleotides can then proceed either by such an oxidisation process, or by the exchange reaction between the thiol groups and the disulphide bonds as illustrated in FIG. 1A;

Secondly, as shown in FIG. 1B, the conjugation of thiol labelled oligonucleotides with (3-aminopropyl)-trimethyoxysilane (NH2-Silane) is enabled by the presence of the heterobifunctional linkers (cross-linking agents) N-succinimidyl-3-(2-pyridyldithiol)-propionate (SPDP), or, succinimidyl-6-(iodoacetyl-amino)-hexanoate (SIAX), in dimethyl sulphoxide; and

Thirdly, with reference to FIG. 1C, acrylic labelled oligonucleotides are conjugated to (3-methacryloxy-propyl)-trimethoxysilane in 0.3 M (pH 3.7) sodium acetate buffer supplemented with ammonium persulphate and N,N,N′,N′-tetramethylenediamide, TEMED, (final concentration of 0.5% and 2%, respectively).

For the purpose of producing chips, the presently preferred pathway is the reaction of thiol labelled oligonucleotides with mercapto silanes, and more preferably with (3-mercaptopropyl)-trimethoxysilane, since it is the fastest and most simple procedure. For preparing oligonucteotide monolayers, however, a reaction or deposition medium should be used, which minimises the evaporation thereof, so that even monolayers of the silanised oligonucleotides readily can be formed. Accordingly, a suitable reaction or deposition medium would, for example, be a DMSO-based one.

As already mentioned, there is a vast variety of different silylating reagents (silanes), many of which are useful in the present invention, and will not be specifically mentioned herein, since the skilled person in the art readily will be able to choose suitable silanes for the specific oligonucleotide to be immobilised, as the case may be, merely by performing simple routine experiments, with reference to the silane chemistry.

Depending on the specific label on the nucleic acid and the group of the silylating reagent used, which group is to be linked together with the label. a coupling agent might be required in order to initiate the attachment of the silyl-group carrying silvlating agent to the label of the nucleic acid. Such coupling agents are well known in the art. Examples of such coupling agents are, for example, 1-ethyl-3-(3-dimethylaminopropyl) carbodiimide-hydrochloride, 1-cyclohexyl-3-(2-morpholino-ethyl) carbodiimide, dicyclohexylcarbodiimide, diisopropyl carbodiimide, N-ethyl-3-phenvlisoxazolium-3′-sulphonate. N,N′-carbonyldiimidazole. As will be realised by the person skilled in the art, the specific choice of coupling agent is also dependent upon the solvent used. For example, in the case of a nucleic acid modified with a carboxyl group, and the silylating agent (3-aminopropyl)-trimethoxysilane, in an aqueous solvent, a suitable coupling agent is for instance 2-ehyl-3-(-dimethylaminopropyl)carbodiimide hydrochloride.

As previously mentioned, in lieu of a coupling agent, bifunctional cross-linking agents can be used. These are also well-known, and will be apparent to the skilled person in the art, having read this disclosure. These can readily be selected by the skilled person based upon the-label (modification) of the nucleic acid and the group (functionality) of the silylating agent to be conjugated with the nucleic acid, and also on the solvent used. Both homo and hetero bifunctional cross-linking agents can be used. Especially if the functionality present on the silylating agent, and the group by which the nucleic acid is modified is the same, a homo functional may be used. As will readily be realised. in such a case, in addition to the desired product, there will probably be some formation of cross-linking agent molecules having one nucleic acid molecule attached at each end thereof, and also of nucleic acid molecules having one silylating agent molecule attached at each end thereof. On the other hand, where a hetero bifunctional cross-linking molecule is used, together with a suitable functionality of the silylating agent, and the nucleic acid, respectively, the desired product will be formed predominantly. Examples of cross-linking agents are dithiobis(succinimidylpropionate), disuccinimidylsuberate, disuccinimidyl tartrate, bis[2(succinimidooxycarbonyloxy)ethyl]sulphonate, ethyleneglycobis (succinimidylsuccinate), disuccinimidyl glutarate, N,N′-isuccinimidyl carbonate, dimethyladipimidate dihydrochloride, dimethylpimelimidate dihydrochloride, dimethylsuberimidate dihydrochloride, dimethyl-3,3′-dithiobispropionimidate, 1,4-di-[3′-(2′-pyridyldithio)-propionamido]-butane, bismaleimidohexane, 1,5-difluoro-2,4-dinitrobenzene, 1,4-butandiol diglycidyl ether, adipicacid dihydrazide, carbohydrazide, N-succinimidyl-3-(2-pyridyldithio)-propionate, succinimidyl-4-(N-maleimidomethyl)-cclohexane-1-carboxylate, m-maleimidobenzoyl-N-hydroxysuccinimide ester, N-succinimidyl-(4-iodoacetyl)-aminobenzoate, succinimidyl-4-(p-maleimidophenyl)-butyrate, succinimidyl-6-[(iodoacetyl)amino]-hexanoate, succinimidyl4[((iodoacetyl)amino)-metyl]-cyclohexane-1-carboxylate, p-nitrophenyliodoacetate, 4-(4-N-maleimido-phenyl)-butyric acid hydrazidehydrochloride, 4-(N-maleimidomethyl)-cyclohexane-1-carboxyl-hydrazidehydrochloride, 3-(2-Pyridyldithio)-propionyl hydrazide.

### EXAMPLES

The invention will now be further illustrated by way of the following examples and the attached drawings. In the examples all chemicals were purchased from Fluka, USA, unless otherwise indicated. All oligonucleotides were purchased from Interactiva, Germany, except for acrylic labelled oligonucleotides which were purchased from Third Wave technologies Inc., USA.

General Procedures Used in the Examples

Glass slides (from Kebo-lab, Sweden) were washed in H₂O in an ultrasonicator for 30 minutes and then treated in 10% NaOH for 30 minutes, followed by 30 minutes in 5% HCl. The slides were then rinsed in tap water, distilled water, and 99.9% ethanol and dried at 80° C. for 1 hour, and thereafter cooled to room temperature and stored at room temperature.

Silanised nucleic acids obtained in the Examples were spotted onto the glass slides either manually (ca. 120 nl/spot) or with an automated arrayer (Genetic Microsystem, USA) (ca. 50 pl/spot). For nucleic acids in aqueous solutions (i.e., from Examples 1, 2 and 4), the glass slides after spotting were allowed to dry at room temperature (ca. 5 min.) and then further dried at 50° C. for 5 min. The slides were then dipped into hot H₂O (90° C.-100° C. for 5 min. to remove any non-covalently bound nucleic acids before proceeding to the hybridisation step given below. For oligonucleotides in DMSO (i.e., from Example 3), the slides were left at room temperature for 10 min. and then dried at 50° C. (10 min.). These slides were sequentially washed in 3×2 min in DMSO, 3×2 min. in ethanol, and then 2×5 min. in hot water (90° C.-100° C.), and then used for hybridisation detection.

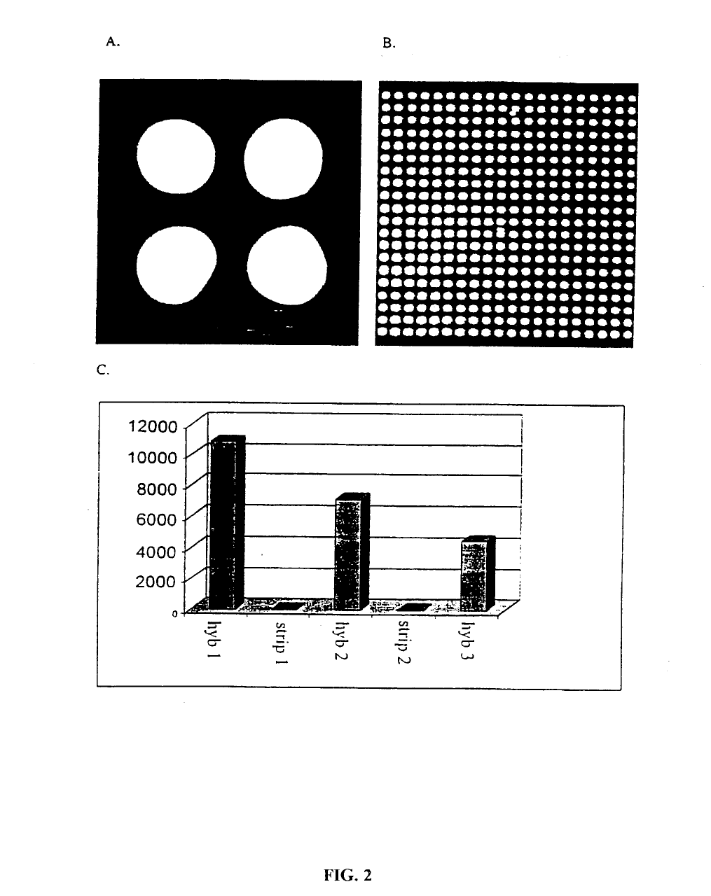

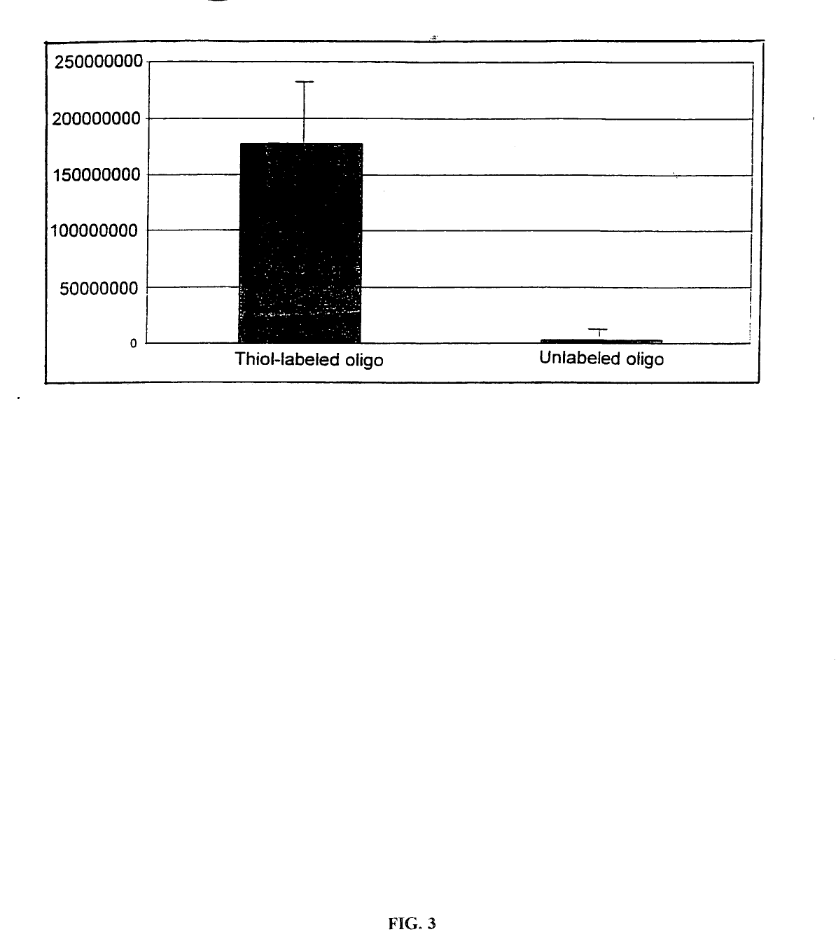

Nucleic acid immobilisation using the silanised nucleic acid method was detected by hybridisation in the following way. Cy3 (Amersham, Parmacia Biotech) labelled oligonucleotide probes (Lac-Cy3, Cy3-5′-GGAAACAGCTATGACCATGA-3′, LacR1-Cy3, Cy3-5′-GCAGGCTTCTGCTTCAATCA-3′) were diluted to 0.02-2 μM in 5×SSCT (750 mM NaCl, 125 mM sodium citrate, pH 7.5, 0.1% Tween-20) and applied on the surface of the glass slides. A glass coverslip was mounted gently on top of the solution. Then the complex was transferred into a humidifier (pre-warmed to 37° C.) in a 37° C. incubator to hybridise for 30 min to overnight. Afterwards the unbound probes were removed by rinsing 3×1 min in 5×SST at room temperature. The slides were further washed 2×15 min in 1×SST (150 mM NaCl, 25 mM sodium citrate, pH 7.5, 0.1% Tween-20) at 37° C. After washing, the slides were dried under nitrogen gas. Hybridisation signal was observed by using a fluorescent scanner (Genetic microsystem, USA) and the results are shown in FIGS. 2, 3, 4, 5, 6, and 7 in the attached drawings.

### Example 1

Conjugation of (3-mercaptopropyl)-methoxvsilane to thiol Labelled oligonucleotides (as shown in FIG. 1A), and Subsequent Immobilisation Thereof

For conjugation of 5′-thiol labelled oligonucleotides, i.e., Lac-thio, having the nucleotide sequence of TCATGGTCATAGCTGTTTCC, and Lac-thio-sen, having the sequence of GGAAACAGCTATGACCATGA, to (3-mercaptopropyl)-trimethoxysilane, a reaction mix containing 50 μM of the oligonucleotides, 250 μM (3-mercaptopropyl)-trimethoxysilane, which was added from a fresh 5 mM solution in 0.3 M (pH 3.7) sodium acetate buffer, was prepared in 0.3 M (pH 3.7) sodium acetate buffer. The reaction can be routinely configured to a volume of 10 μl-200 μl. In this example, a total volume of 20 μl was used. The reaction was allowed to proceed for at least 10 minutes, with a normal time range of 10 min. to 2 hours at room temperature. Thereafter, the reaction mix can be directly used or diluted with 0.3 M (pH 3.7) sodium acetate buffer to the desired concentration of the conjugated oligonucleotides for immobilization on glass surface.

The conjugated molecules obtained above were deposited onto pre-cleaned glass slides and the chips were further treated as specified above in the General procedures. Then the slides were hybridise with Lac-Cy3 to detect covalent immobilisation of Lac-thio. As shown in FIGS. 2A, and 2B, the conjugated molecules did get immobilised on glass surfaces and were available for hybridisation. With the fact that the 50 pl droplets delivered on glass surface actually evaporated completely within 5-10 seconds, the results demonstrated that the silane moiety conjugated on oligonucleotides reacts readily with glass and results in the covalent crosslinking in seconds. To further illustrate the covalent nature of crosslinking between the silanised oligonucleotides and the glass chips, the glass chips were stripped and re-hybridise with the fluorescent probe Lac-Cy3 3 rounds. Under identical hybridisation conditions (probe concentration 2 JIM, at 37° C. for 30 min), after 3 rounds of hybridisation/stripping, there were sufficiently high levels of oligonucleotides attached on the chip to achieve a 1000 times higher than background signal (FIG. 2C). This confirms that the immobilisation process employed here does result in very durable covalent bonds between the nucleic acid moiety and glass surface.

We also noticed marked reduction of signals during successive stripping and hybridisation (FIG. 2C). The mechanism by which such decreases could be obtained is not yet fully understood, but it is believed to be the consequence of the disruption of disuiphide bonds at high temperature, i.e., breakage of the conjugates.

With reference to FIG. 3, to demonstrate the specificity of the reaction between thiol $groups, or between thiol and disulfide bonds, (3-mercaptopropyl)-trimethoxysilane incubated was incubated with equal molar amounts of oligonucleotides containing either a thiol group, or no label. After spotting the samples to glass surface and routine washing, the chip was hybridise to Lac-Cy3 to determine the correlation of oligonucleotide immobilisation with the labelling of oligonucleotides. It was demonstrated that oligonucleotides without any modification did not become immobilised significantly (less than 2% of the signal from thiol modified oligonucleotides). These results suggest that the nucleic acid backbone does not contribute to the hiah level of immobilisation observed with thiol oligonucleotides, and the reaction is mostly thiol specific.

### Example 2

Conjugation of (3-mercaptopropyl)methoxysilane to thiol Labelled DNA Fragment Obtained by PCR, and Immobilisation Thereof.

A thiol labelled 1-kb LacZ DNA fragment was generated by using one thiol labelled primer (Lac-thio-sen) with an unlabelled reverse primer (LacR1, GCAGGCTTCTGCTTCAATCA) in 50 μl volume in 1×PCR buffer (Amnersham Pharmacia Biotech) supplemented with 2.5 mM MaCl₂, and 100 M of each of DATP, dTTP, dCTP, and dGTP (final concentration for all). The PCR product was either directly used for subsequent conjugation reaction, or precipitated and then re-suspended in 10 μl H₂O (5 fold concentration) before proceeding to the conjugation step. 8 μl of the original PCR mix or concentrated product was combined with 250 μM (3-mercaptopropyl)-trimethoxysilane and 1 μl 3 M (pH 3.7) sodium acetate buffer in 10 μl final volume and allowed to react for 2 hours.

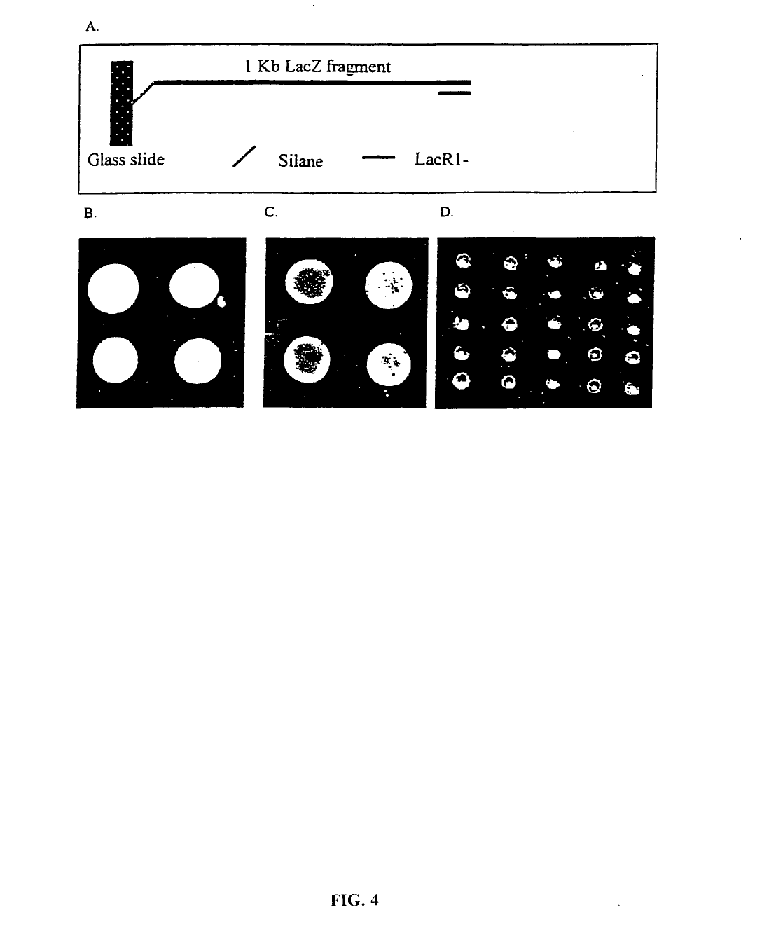

Thus, PCR was performed on a LacZ cDNA fragment using Lac-Thio-sen and LacR1 so that one strand of the cDNA is thiol labelled. The PCR products were conjugated to (3-imercaptopropyl)-trimethoxysilane with (FIG. 4B) or without (FIGS. 4C and 4D) 5 folds concentration by precipitation. Then the silanised CDNA was spotted onto clean glass chips, and the chips were treated as specified in the general procedures. Then the immobilisation was visualised by hybridising to a nucleotide probe complementary to the very distal end of the attached cDNA strand as shown in FIG. 4A. The results demonstrated that the method can result in rapid immobilisation of CDNA as well (FIGS. 4B, C, and D). The CDNA chips were stripped by boiling in water for 1 minute and re-hybridise with the same probe, and the result suggested that even with cDNA chips where hydrolysis of long cDNA chains during prolonged incubation could be phenomenal, reusing of such chips is still a possibility (FIG. 4C).

### Example 3

Conjugation of (3-aminopropyl)-trimethyoxysilane to thiol Labelled oligonucleotides Using Linkers (as shown in FIG. 1B)

Conjugation of Lac-thio and Lac-thio-sen with (3-aminopropyl)-trimethyoxysilane (hereinafter NH₂-Silane) in the presence of heterobifunctional linkers (N-succinimidyl-3-(2-pyridyldithiol)-propionate (SPDP), and succinimidyl-6-(iodoacetyl-amino)-hexanoate (SIAX), respectively), was performed in DMSO. The oligonucleotides were first solubilised in H₂O at 1 mM concentration. Then different volumes of the 1 mM stock solution were combined with 250 μM NH₂-Silane (added from 5 mnM solution in ethanol) and 250 μM bifunctional reagents (added from 5 mM stock solution in DMSO) in DMSO. The reaction was allowed to proceed for 1-2 hours at room temperature. The final concentration of silane conjugated oligonucleotides was from 5 μM to 50 μM.

It is common knowledge that silanes are unstable in aqueous solutions, especially at high pH. With the low pH buffer used in our system, the degradation of silane is minimized, but not eliminated. Among the arsenal of silanes and bifunctional reagents that can crosslink silane and a third party molecule, many reagents are not water soluble at all (like SPDP and SIAX). One ideal situation would be to configure the conjugation reaction in an organic solvent to maximise the integrity of the silane moiety or to accommodate those non water-soluble reagents. This can, for example, be done by carrying out the conjugation of thiol oligonucleotides with an amino silane in dimethyl sulphoxide in the presence of SPDP or SIAX (FIG. 1B). Oligonulceotides can not be directly solubilised in DMSO, and this problem can be overcome by first making up a concentrated oligonucleotide solution in H₂O, and then diluting this stock solution into DMSO. The conjugated oligonucleotides were directly spotted onto glass chips in DMSO. After successive washing in DMSO, ethanol and water, the chips were evaluated by hybridisation. It was shown that a reaction system in organic solvent is feasible and can result in covalent immobilisation of DNA molecules. Oligonucleotides in DMSO solution were also spotted using an automated arrayer (Genetic Microsystems, USA) and the chips were tested by hybridisation. It was shown that an organic solvent like DMSO is also compatible with the commercial arrayer. It was noted that the uniformity of delivery of DMSO solution by the arrayer is not as uniform as that of aqueous solution.

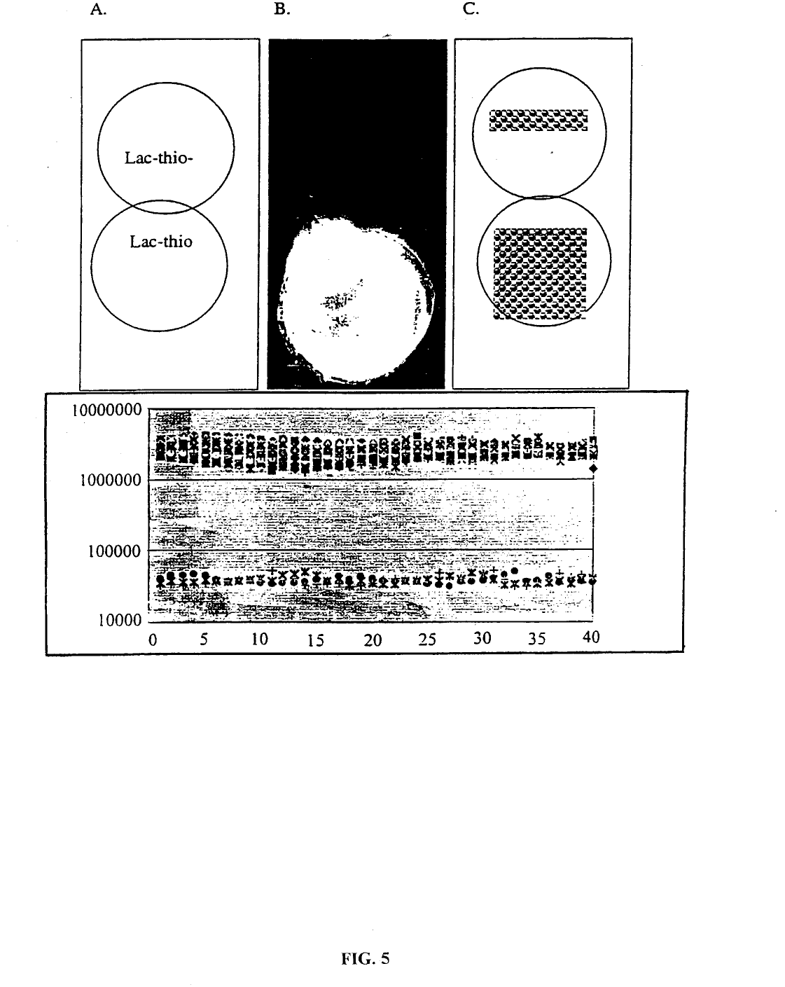

The silanised oligonucleotides in DMSO were used to exploit the formation of DNA monolayers on glass chips. Such monolayers would be very useful for fabricating DNA based biosensors. 10 μl of 20 μM Lac-thio and Lac-thio-sen were spotted on glass slides and covered by 16 mm round coverslipes, as shown in FIG. 5A. After 30 min the coverslips and the DMSO solution were rinsed off by dipping the chips into DMSO for 3×2 min, in ethanol for 2-3 min., and in 90° C. water for 5 minutes. The oligonucleotide monolayers formed were evaluated by hybridisation to LacCy3 (FIG. 5B). The uniformity of the monolayer was further analysed using ImageQuant (from Molecular Dynamics, USA) by sampling 1600 spots from the Lac-thio coated area as shown in FIG. 5C. The intensity of the 1600 spots from the Lac-thio monolayer and 120 spots randomly sampled from a control Lac-thio-sen monolayer were plotted in 40 spot-rows (FIG. 5C). Statistics data showed that the monolayer is quite uniform and the standard deviation of the intensity across the 1600 spots was only 16% of the signal, whereas the signal from a sense control oligonucleotide (lac-thio-sen) was more than 80 folds lower (FIG. 5D).

### Example 4

Conjugation of (3-methacryloxy-propyl)-trimethoxysilane to acrylic Labelled oligonucleotides (as shown in FIG. 1C) and Immobilisation Thereof.

Acrylic oligonucleotides (Lac-acrylic: acrylic-5′-TCATGGTCATAGCTGTTTCC-3′, Lac-acrylic-sen: acrylic-5′-GGAAACAGCTATGACCATGA-3′) (5-50 μM) were combined with 250 μM acrylic silane in 0.3 M (pH 3.7) sodium acetate buffer. Ammonium persulfate and N,N,N′,N′-tetramethylenediamide, TEMED, were added to a final concentration of 0.5% and 2%, respectively. The mixture was allowed to polymerise for 30 min at room temperature, thereby obtaining the desired conjugates of (3-methacryloxy-propyl)-trimethoxysilane to acrylic oligonucleotide.

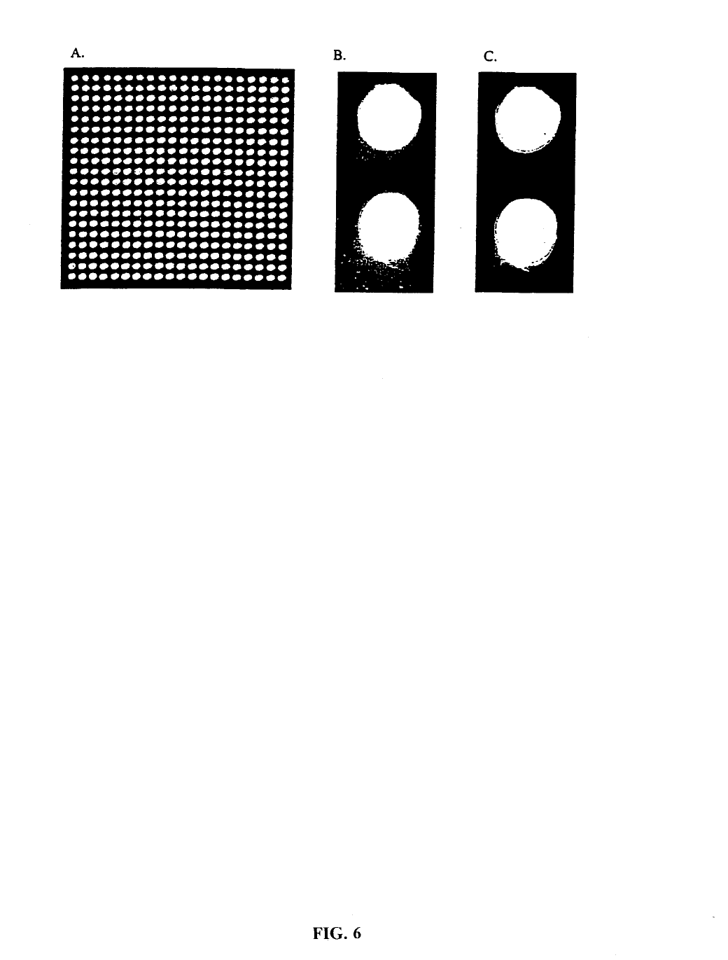

This is an alternative pathway that was tested to conjugate a silyl moiety to oligonucleotides by crosslinking acrylic oligonucelotides with an acrylic silane in solution in the presence of ammonium persulfate and TEMED (FIG. 1C). The conjugated molecules were spotted and the chips were treated as specified in the general procedures. Then the immobilisation of nucleic acids was visualised by hybridising to Lac-Cy3 and fluorescent detection (FIGS. 6A and 6B). After stripping with boiling water, the chips were re-hybridised with the same probe, as shown in FIG. 6C, and comparable level of signal was observed.

Re-Silanization of the DNA Chips

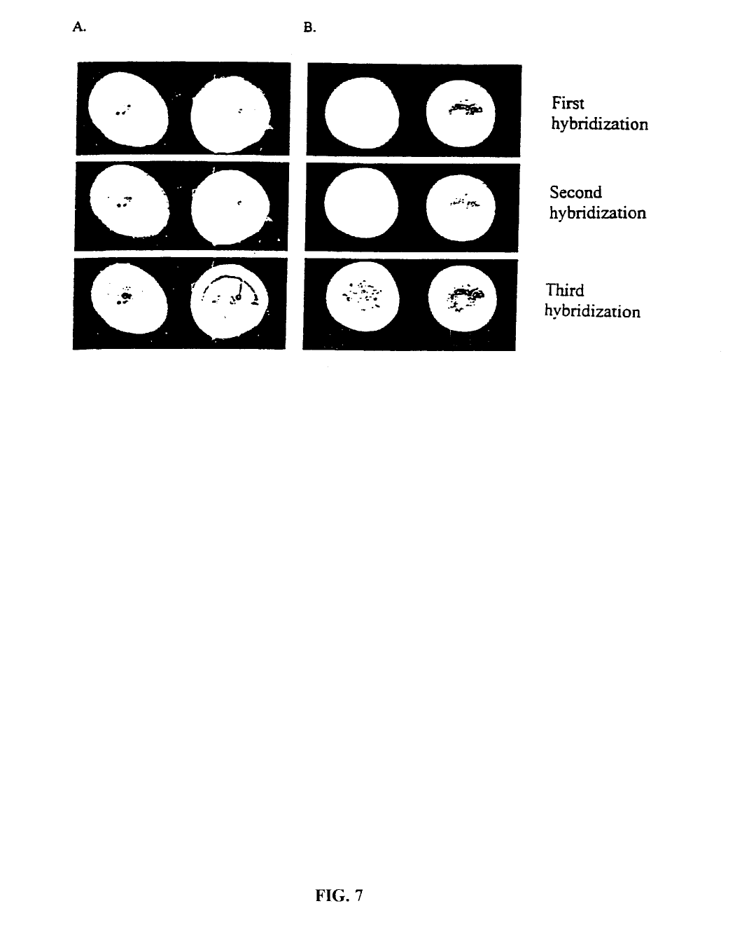

DNA chips produced using methods described herein were not surface-modified at all in areas that is not covered by DNA spots. These uncovered areas can then accommodate the deposition of additional molecules via different silane reagents. We demonstrated that dimethyl-dichlorosilane can be used to modify those areas to create an overall hydrophobic surface around the DNA spots without interfering with the accessibility of DNA on the chips, as shown in FIG. 7, A. The advantage of treating chips with a hydrophobic silane is that the washing and drying of the chips are greatly facilitated because the solution carryover is largely diminished. Drying time was reduced by a factor of 5-10. According to the prior art, re-silanised chips can be generated by chemically modifying chips produced on globally silanised glass surface, however, the approach according to the present invention facilitates the achievement of this effect. The ease of handling re-silanized chips according to the present invention is definitely an advantage in high throughput applications.

1. Method of immobilising nucleic acid in solution onto glass and silicon surfaces by applying said solution onto said surfaces, c h a r a c t e r i s e d in that said nucleic acid is silylated by means of a silylating reagent, optionally in the presence of a coupling agent or a bifunctional cross-linking agent, so as to exhibit a reactive silyl group capable of being immobilised onto unmodified glass and other silicon surfaces.

2. Method of claim 1, wherein said nucleic acid is at least one member selected from the group consisting of oligonucleotide, DNA, RNA, PNA, and any fractions or modifications thereof.

3. Method of claim 1 or 2, wherein said nucleic acid is modified at least at one end thereof by a reactive functionality, preferably selected from thio-, dithio-, amino-, aldehydo-, keto-, hydrazo-, acrylic-, hydrazino-, or halo-, or any other functionality having the capability of reacting with said silylating reagent, optionally in the presence of a coupling agent or bifunctional cross-linking agent.

4. Method of any of the preceding claims, wherein said silylating reagent comprises a reactive group, preferably selected from amino, mercapto, thio, acrylic, cyano, glycidoxy, or any other reactive group capable of reacting with the reactive functionality of the nucleic acid, optionally in the presence of a coupling reagent, or with at least one of the reactive groups of a cross-linking agent.

5. Method of any of the preceding claims, wherein said coupling agent is selected from the group of 1-ethyl-3-(3-dimethylaminopropyl) carbodiimide-hydrochloride, 1-cyclohexyl-3-(2-morpholino-ethyl) carbodiimide, dicyclohexylcarbodi-imide, diisopropyl carbodiimide, N-ethyl-3-phenylisoxazolium-3′-sulphonate, N,N′-carbonyldiimidazole.

6. Method of any of the preceding claims, wherein said bifunctional cross-linking agent is selected from dithiobis(succinimidylpropionate), disuccinimidylsuberate, disuccinimidyl tartrate, bis[2(succinimidooxycarbonyloxy)ethyl]sulphonate, ethyleneglycobis (succinimidylsuccinate), disuccinimidyl glutarate, N,N′-disucciniimidyl carbonate, dimethyladipimidate dihydrochioride, dimethylpimelimidate dihydrochloride, dimethylsuberimidate dihydrochloride, dimethyl-3,3′-dthiobispropionimidate, 1,4di-[3′-(2′-pyridyldithio propionamido]-butane, bismaleimidohexane, 1,5-difluoro-2,4-dinitrobenzene, 1,4-butandiol diglycidyl ether, adipicacid dihydrazide, carbohydrazide, N-succinimidyl-3-(2-pyridyldithio)-propionate, succinimidyl-4-(N-maleimidomethyl)-cyclohexane-1-carboxylate, m-maleimidobenzoyl-N-hydroxysuccinimide ester, N-succinimidyl-(4-iodoacetyl)-aminobenzoate, succinimidyl-4(p-maleimidophenyl)-butyrate, succinimidyl-6-[(iodoacetyl)amino]-hexanoate, succinimidyl-4[((iodoacetyl)-amino)-metyl]-cyclohexane-1-carboxylate, p-nitrophenyliodoacetate, 4-(4-N-maleimido-phenyl)-butyric acid hydrazidehydrochloride, 4-(N-maleimidomethyl)-cyclohexane-1-carboxyl-hydrazidehydrochloride, 3-(2-Pyridyldithio)-propionyl hydrazide.

7. Method of any of the preceding claims, wherein the solvent is selected from the group of water, DMSO, DMF, and aqueous buffer solutions, or any combination thereof.

8. Silanised nucleic acid which can be used in the method of claim 1, and which is obtained by silylating a nucleic acid by means of a silylating reagent, optionally in the presence of a coupling agent or a bifunctional cross-linking agent.

9. The use of the method of any of the preceding claims for the preparation of microarrays.

10. Glass or silicon chips or slides having silanised nucleic acids immobilised thereto, optionally in a monolayer, obtainable according to the method of claim 1.
